(19) United States
(12) Patent Application Publication
(10) Pub. No.: US 2017/0035360 A1
(43) Pub. Date: Feb. 9, 2017

(54) BIOMETRIC PARAMETER DATA EXTRACTION FROM A PATIENT SURFACE BY AIR PRESSURE SENSING

(71) Applicant: Hill-Rom Services, Inc., Batesville, IN (US)

(72) Inventors: Gavin M. Monson, Oxford, OH (US); John D. Christie, Batesville, IN (US); Gregory John Shannon, Indianapolis, IN (US); John Goewert, Batesville, IN (US); John G. Byers, Batesville, IN (US); Dan R. Tallent, Hope, IN (US); Douglas A. Seim, Okeanna, OH (US)

(21) Appl. No.: 15/221,681

(22) Filed: Jul. 28, 2016

Related U.S. Application Data
(60) Provisional application No. 62/201,324, filed on Aug. 5, 2015.

(51) Int. Cl. A61B 5/00; A61B 5/11; A61B 5/113; A61B 5/0205

(52) U.S. Cl. CPC A61B 5/6892; A61B 5/0205; A61B 5/1102; A61B 5/113; A61B 5/742; A61B 5/7475; A61B 5/7203; A61B 5/7257; A61B 5/726; A61B 5/725; A61B 2562/0247; A61B 2562/028; A61B 2562/168

(57) ABSTRACT

In a method or system for obtaining patient biometric parameter data from a patient support surface comprising at least one air pressure bladder inflated by air pressure through an air supply line from an air pump reservoir controlled by a bladder air controller receiving an air pressure signal from a pressure sensor in the air supply line, signal processing a variation of the air pressure signal from the pressure sensor to extract the patient biometric parameter data.

### BACKGROUND

It is known to obtain biometric parameter data from a patient surface supporting a patient by use of one or more force sensors which record a force applied by the patient's body surface on the sensor. The patient support surface has a plurality of bladders where pressure data is measured to control the amount of air pressure applied to the bladder. See for example U.S. Pat. No. 7,515,059. Also see U.S. Pat. No. 7,699,784 where the signals output by one or more force sensors are analyzed, such as by a Fast Fourier Transform analysis.

In U.S. Pat. No. 8,413,273 a hospital bed chair is shown with an inflatable bladder wherein a weight of the patient can be measured by measuring the force imparted by the bladder to a load cell (force sensor).

In US Patent Publication 2014/0135635 one or more force transducers are provided for a patient support apparatus. A signal processing is applied to the signals output by the force transducers to determine patient parameters such as blood volume, heart rate, and respiratory rate information. Heart rate and respiratory rate information is derived from the blood volume pulse information. The signal processing to accomplish such extraction is shown in FIG. 5 of the '635 publication and includes the use of Fast Fourier Transform power spectrum analysis, bandpass filters, and power spectral density analysis.

### SUMMARY

It is an object to utilize a patient support surface to obtain patient biometric parameter data without the use of force sensors impacted by a patient support surface.

In a method or system for obtaining patient biometric parameter data from a patient support surface comprising at least one air pressure bladder inflated by air pressure through an air supply line from an air pump reservoir controlled by a bladder air controller receiving an air pressure signal from a pressure sensor in the air supply line, signal processing a variation of the air pressure signal from the pressure sensor to extract the patient biometric parameter data.

### BRIEF DESCRIPTION OF THE DRAWINGS

FIG. 1 is a perspective view of a system for obtaining patient biometric parameter data by measuring air pressure signal variation in at least one bladder of a patient support surface; and

FIG. 2 is a diagram of a patient biometric parameter data extraction processor and more particularly illustrating functional blocks within the processor for extraction of patient biometric parameter data based on variation of at least one air pressure signal.

### DESCRIPTION OF EXEMPLARY EMBODIMENTS

For the purposes of promoting an understanding of the principles of the invention, reference will now be made to preferred exemplary embodiments/best mode illustrated in the drawings and specific language will be used to describe the same. It will nevertheless be understood that no limitation of the scope of the invention is thereby intended, and such alterations and further modifications in the illustrated embodiments and such further applications of the principles of the invention as illustrated as would normally occur to one skilled in the art to which the invention relates are included herein.

Extraction of key biometric indicators passively from the patient environment is an area that is of increasing importance. The present exemplary embodiment addresses a method of extracting biometric indicators from a patient including but not limited to heart rate and breathing rate of a patient using hardware which may already be present in a hospital bed.

According to one exemplary embodiment, a novel method and apparatus is provided of extracting patient biometric data from one or more existing pressure sensors used to control pressure in at least one bladder of a patient support surface to allow better data on a patient's state of health with substantially no additional hardware and using primarily software. Data such as heart rate, breathing rate, or other body sounds can be harvested and analyzed using advance signal processing algorithms to construct a state vector for the patient.

The present exemplary embodiment uses pneumatic pressure sensors already employed to regulate the air pressure inside the patient support surface, colloquially called the mattress. These pressure sensors are typically MEM (micro electro-mechanical system) piezo load beam types of sensors, which have a wide bandwidth output (relative to the bandwidth of the pressure actually being monitored) allowing higher bandwidth signals which emanate from the patient and are impressed onto the surface to be sensed as a small signal perturbation on the much larger, relatively static pressure signal. One commercially available sensor has a 1 kHz sensor bandwidth for zero to full scale pressure readings. Frequency response for smaller amplitude signals is not specified, but is likely even higher. Still, a 1 kHz sensor bandwidth is enough to capture breathing and heart rates, as well as breath sound and cardiac sound capture for audio spectral analysis and anomaly detection. Other sounds produced by the human body may be able to be sensed and processed such as bowel sounds to facilitate early detection of problem conditions developing in patients. Patient motion can be inferred using characterization algorithms to spot pressure variations associated with patient movement.

In the exemplary embodiment the mechanical connection that the surface enclosure has to the volume of air used to support the patient is employed to couple these sounds in the form of pressure fluctuations to the MEMS pressure sensors, which represent these pressure waves as small-signal disturbances around a quiescent point which has variable signal to noise ratio, dependent upon the relative magnitude of the pressure signal which is quasi-static and the biometric signals which are much lower amplitude but higher frequency.

Biometric data capture is not limited to simply air pressure sensors. If other sensors (accels, magnetometers, gyroscopes, electric field sensors, temperature sensors, etc.) are included, then there are myriad biometric data that can be harvested and analyzed to get a better picture of the overall health of the patient.

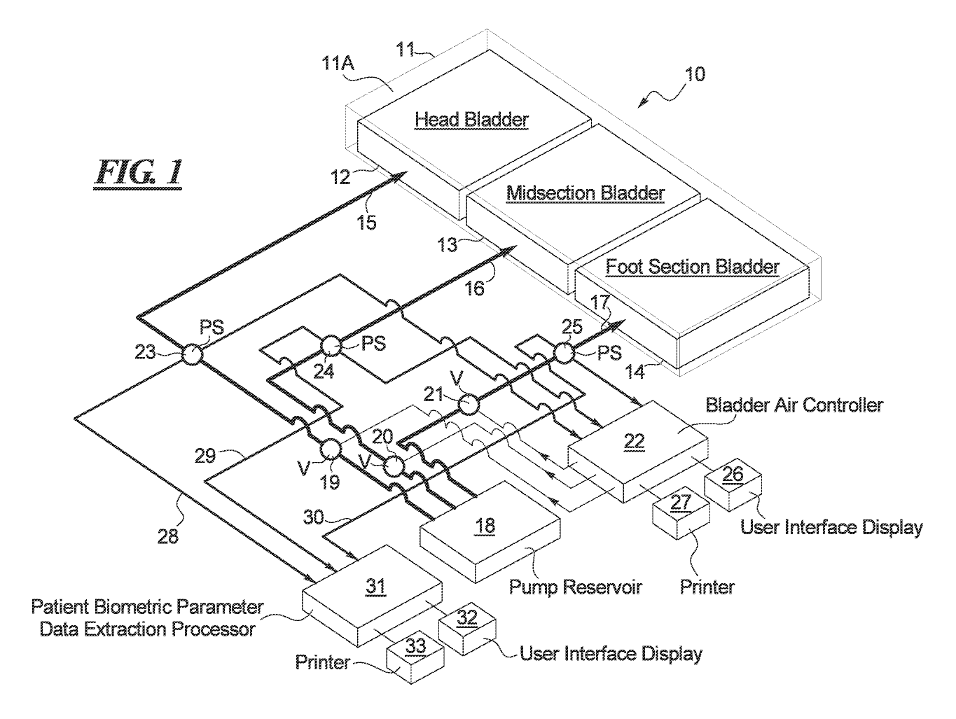

As shown in FIG. 1, a patient support surface 10 comprises an outer enclosure 11 having a top surface 11A on which the patient rests. Within the other enclosure 11 a plurality of bladders including a head bladder 12, a midsection bladder 13, and a foot section bladder 14 may be provided. These bladders are inflatable by air pressure. The air pressure is supplied to each of the respective bladders 12, 13, 14, by respective air supply lines 15, 16, and 17 from an air pump reservoir 18. Air pressure is controlled via respective valves 19, 20, and 21 controlled by a bladder air controller 22. The bladder air controller 22 receives a respective air pressure signal from respective pressure sensors 23, 24, and 25. A display device 26 and a printer 27 are preferably connected to the bladder air controller 22 for displaying bladder pressure data for each of the respective bladders.

In one exemplary embodiment, the bladder air control system described above associated with the patient support surface 10 may be pre-existing and already installed, such as for a patient hospital bed. With the exemplary embodiment, the pressure sensors 23, 24, and 25 are also utilized to provide respective air pressure signals via electrical lines 28, 29, and 30 to a patient biometric parameter data extraction processor 31 which determines the patient biometric parameter data based on signal processing of a respective variation of the respective air pressure signals. This data extraction processor 31 is preferably connected to a respective display 32 and respective printer 33 for output of extracted patient biometric data for various patient biometric parameters. Furthermore, as previously described, preferably the pressure sensors, which may be pre-existing, have a 1 kHz sensor bandwidth which is sufficient to capture breathing and heart rates as well as breath sound and cardiac sound capture for audio spectral analysis and anomaly detection. As previously indicated these are known as MEMS pressure sensors (micro electro-mechanical system) and piezo load beam types of sensors.

Thus with the method and system of the exemplary embodiment, extraction of patient biometric data can be performed from existing surface pressure sensors to allow better data on a patient's state of health with perhaps no additional hardware and perhaps employing software only.

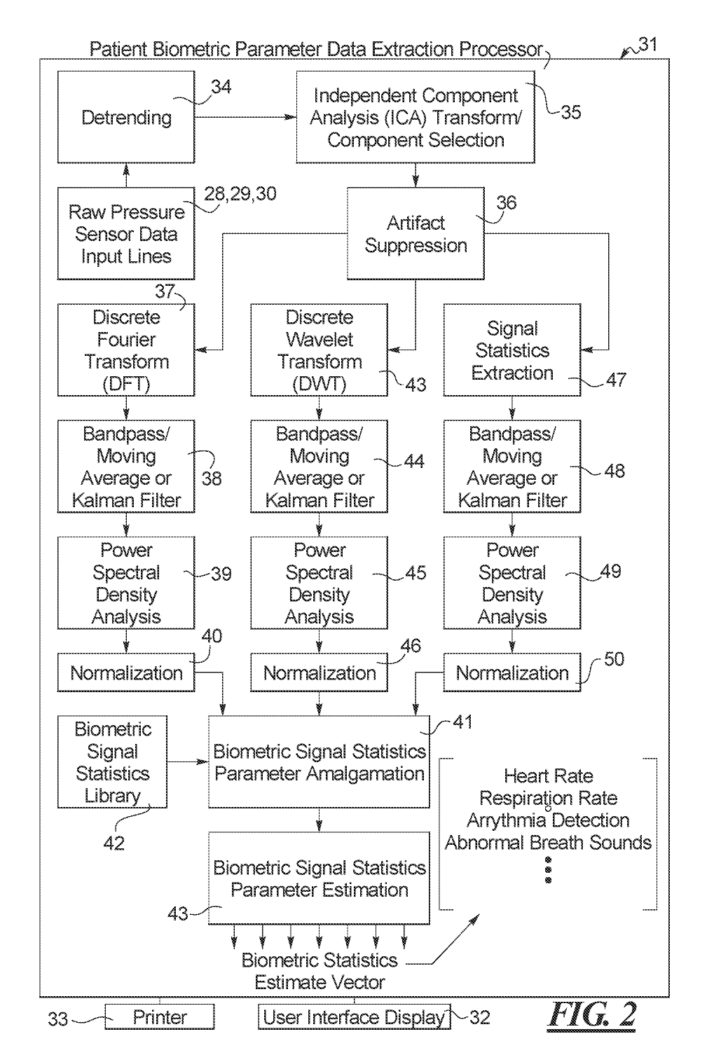

FIG. 2 illustrates in greater detail the patient biometric parameter data extraction processor 31 illustrated in FIG. 1 whereby data such as heart rate, breath rate, and other body sounds can be harvested and analyzed using advanced signal processing algorithms to construct a biometric parameter estimate vector for the patient. Thus acoustic signature signals can be used to determined biometric signals.

Each of the functional blocks illustrated in FIG. 2 in the extraction processor 31 will now be described. These functional blocks are preferably software. Detrending block 34 receives the raw pressure sensor data on the pressure signal supply lines 28, 29, and 30. These pressure signals are preferably 0-5 volts typically. The data may be collected at a rate of 15 Hz, for example. The detrending block 34 processes the data to remove offset and/or gain errors and/or for detrending artifacts in the data (in one exemplary embodiment smoothing of data). The output of the data that has undergone detrending in block 34 is preferably a 24 bit signal although the signal may be of any size and frequency. The detrending is accomplished through normalization based on a smoothness priors approach as shown in Equation 1 below. In this equation, yᵢ(t) is the raw load cell signal, μᵢ and Ωᵢ are the mean and standard deviation of signal yᵢ(t) respectively and yᵢ′(t) is the normalized signal for each source of signal i=1, 2, 3, . . . , n.

$${y}_{i}^{′} ( t ) = \frac{{y}_{i} ( t ) - {μ}_{i}}{{σ}_{i}} \qquad Equation 1$$

In other exemplary embodiments other methods of normalizing the data may be employed.

In block 35 for independent component analysis (ICA) transform/component selection, a blind source separation operation is performed using an independent component analysis (ICA) transform. ICA is one technique for separation of independent signals from a set of observations that are composed of linear mixtures of underlying source signals. The underlying signal of interest in this embodiment is the blood volume pulse information (BVP) that propagates through the body. During the cardiac cycle, increased flow through the body's blood vessels results in forces produced by the body on to objects in contact or in proximity to the body. As the BVP changes, the bladder pressure sensors record a mixture of the BVP signal with different weights. These observed signals are denoted by yᵢ′(t), y₂′(t) yₙ′(t) which are signals recorded at time t. In this embodiment the ICA model assumes that the observed signals are linear mixes of the source signals as shown in equation 2 below. In Equation 2 below y′(t) represents a matrix of observed signals, x(t) is an estimate of the underlying source signals and matrix A contains mixture coefficients.

y′(t)=Ax(t) Equation 2

The object of ICA in this embodiment is to determine a demixing matrix W shown in equation 3 below that is an approximation of the inverse of the original mixing matrix A whose output is an estimate of the matrix x(t) containing the underlying source signals. In one embodiment iterative methods are used to maximize or minimize a cost function that measures the non-Gaussianity of each source to uncover the independent sources. In this embodiment ICA analysis is based on the Joint Approximation Diagonalization of Eigenmatrices (JADE) algorithm.

\hat{x}y′(t)=Wy′(t) Equation 3

Blind source separation ICA analysis in operation block 35 is configured to separate from the pressure signals fluctuations caused predominantly by BVP. In one embodiment data received from the pressure sensors include operation of the support surface 10 including but not limited to percussion and vibration therapy and/or inflation and deflation of the surface 10 and/or operation of any motors on the surface and blind source separation ICA analysis is configured to separate these source signals. In one alternate embodiment identification of components of signals indicative of operation of the surface is aided by predetermined information identifying characteristics of operation of the surface 10. The signal of interest identified in operation block 35 undergoes an artifact suppression process in operation block 36 in this embodiment. The artifact suppression operation block in this embodiment includes interpolation and/or removal of data while in another embodiment data may be normalized after interpolation and/or removal. In yet another embodiment operation block 36 for artifact suppression may be omitted.

Once the ICA signals are generated from operation block 35, the function responsible for BVP is uncovered in discrete fourier transform (DFT) operation block 37 upon generation of power spectrums for the ICA signals. In one embodiment the power spectrum of the signal with the highest peak is selected for analysis. In another embodiment the signal with a peak in power spectrum in the range where BVP is known to exist is selected. This is done automatically in this embodiment. However in other embodiments a caregiver may select a signal of interest using the user interface display 32. In one embodiment, weight determined by the pressure sensors is used to determine if a patient is indeed on the support surface 10. If it is determined that a person is not on the support surface 10 the operation is terminated and a message is displayed to the caregiver in another embodiment.

In operation block (bandpass moving average or Kalman filter) 38 the signal of interest is smoothed. In this embodiment the signal of interest is smoothed using a five point moving average filter and bandpass filter in an area of interest, in this embodiment 0.7-4 Hz. In other embodiments any data manipulation algorithm may be used.

In operation block 39 power spectral density estimation analysis of IBI information is used to identify heat rate variability (HRV) information. In this embodiment a Lomb periodogram is used to analyze HRV.

Information from power spectral density estimation analysis is normalized in normalization operation block 40 in this embodiment, while in other embodiments this operation may be eliminated.

Normalized information from normalization operation block 40 is used in the biometric signal statistics parameter amalgamation operation block 41 which also receives biometric signal statistics from library 42.

Operation block 41 outputs the parameter amalgamation to biometric signal statistics parameter estimation operation block 43 which in turn outputs biometric parameter estimate vectors.

The artifact suppression operation block 36 also outputs to operation block 43 (discrete wavelet transform (DWT)) which in turn outputs to bandpass/moving average or Kalman filter operation block 44, power spectral density analysis block 45, and normalization block 46. The blocks 44, 45, and 46 were previously described in connection with operation blocks 38, 39, and 40. The normalization block 46 outputs to the biometric signal statistics parameter amalgamation block 41 and biometric signal statistics parameter estimation block 43 as previously described.

The artifact suppression block 36 also outputs to signal statistics extraction block 47 which in turn outputs to bandpass/moving average or Kalman filter block 48, power spectral density analysis block 49, and normalization block 50. Blocks 48, 49, and 50 are similar to the description previously provided for blocks 38, 39, and 40. Normalization block 50 outputs to the biometric signal statistics parameter amalgamation block 41 and biometric signal statistics parameter estimation block 43 previously described.

Although preferred exemplary embodiments are shown and described in detail in the drawings and in the preceding specification, they should be viewed as purely exemplary and not as limiting the invention. It is noted that only preferred exemplary embodiments are shown and described, and all variations and modifications that presently or in the future lie within the protective scope of the invention should be protected.

We claim as our invention:

1. A method for obtaining patient biometric parameter data from a patient support surface comprising at least one air pressure bladder inflated by air pressure through an air supply line from an air pump reservoir controlled by a bladder air controller receiving an air pressure signal from a pressure sensor in the air supply line, comprising the step of:
signal processing a variation of the air pressure signal from the pressure sensor to extract the patient biometric parameter data.

2. The method of claim 1 wherein a head bladder, a midsection bladder, and a foot section bladder are provided in said patient support surface, and respective pressure sensors being connected in supply lines to each of the bladders from the bladder air controller, and wherein a respective pressure signal is provided by each of the three pressure sensors for the signal processing.

3. The method of claim 1 wherein the pressure sensor has at least a 1 kHz bandwidth.

4. The method of claim 1 wherein the pressure sensor comprises a micro electro-mechanical system piezo load beam type sensor.

5. The method of claim 1 wherein the biometric patient parameter data comprises data at least for patient parameters heart rate and respiration.

6. The method of claim 1 wherein a respective control valve is provided connected in series with the pressure sensor between the bladder air controller and the bladder.

7. The method of claim 1 wherein a user interface display is used to display the patient biometric parameter data.

8. The method of claim 1 wherein the pressure sensor pre-exists at the patient surface prior to connecting a patient biometric parameter data extraction processor to the pressure sensor for the signal processing of the variation of the air pressure signal.

9. The method of claim 1 wherein the signal processing comprises a detrending to at least one of remove offset, remove gain and errors, and detrend artifacts.

10. The method of claim 9 wherein after the detrending a blind source separation operation is performed using an independent component analysis transform.

11. The method of claim 10 wherein after utilizing the independent component analysis transform an artifact suppression operation is performed.

12. The method of claim 11 wherein after the artifact suppression at least one of a discrete fourier transform, a discrete wavelet transform, and a signal statistics extraction operation is performed.

13. The method of claim 12 wherein after at least one of the discrete fourier transform, discrete wavelet transform, and signal statistics extraction operation is performed a bandpass/moving average or Kalman filtering operation smoothes a signal followed by a power spectral density analysis operation and normalization operation.

14. The method of claim 13 wherein following the normalization a biometric signal statistics parameter amalgamation operation is performed resulting in a biometric signal statistics parameter estimation operation and outputting of a biometric statistics estimate factor.

15. A method for obtaining patient biometric parameter data from a pre-existing patient support surface comprising at least one air pressure bladder inflated by air pressure through an air supply line from a pre-existing air pump reservoir controlled by a bladder air controller receiving an air pressure signal from a pressure sensor in the air supply line, comprising the step of:
connecting a signal processor to receive said air pressure signal from the pressure sensor; and
signal processing a variation of the air pressure signal from the pressure sensor by use of the signal processor to extract the patient biometric parameter data.

16. A system for obtaining patient biometric parameter data, comprising:
a patient support surface comprising at least one air pressure bladder inflated by air pressure through an air supply line from an air pump reservoir controlled by a bladder air controller receiving an air pressure signal from a pressure sensor in said air supply line; and
a patient biometric parameter data extraction processor also receiving said air pressure signal and determining the patient biometric parameter data based on a signal processing of a variation of the air pressure signal from the pressure sensor.

17. The system of claim 16 wherein the patient support surface comprises a head bladder, a midsection bladder, and a foot section bladder, and wherein respective pressure signals are connected by supply lines to each of the bladders from the bladder air controller, and a respective pressure signal is provided by each of respective pressure sensors in the respective supply lines to the data extraction processor.

18. The system of claim 16 wherein the pressure sensor has at least a 1 kHz bandwith.

19. The system of claim 16 wherein the pressure sensor comprises a micro electro-mechanical system piezo load beam type sensor.

20. The system of claim 16 wherein the biometric patient parameter data comprises data at least for patient parameters heart rate and respiration.

21. The system of claim 16 wherein a respective control valve is connected in series with the pressure sensor between the bladder air controller and the bladder.

22. The system of claim 16 wherein a user interface display is associated with the patient biometric parameter data extraction processor.

23. The system of claim 16 wherein the patient biometric parameter data extraction processor comprises detrending, artifact suppression, and at least one of discrete fourier transform, discrete wavelet transform, and signal statistics extraction operations.

24. The system of claim 23 wherein the patient biometric parameter extraction processor following at least one of the discrete fourier transform, discrete wavelet transform, and signal statistics extraction operations performs a bandpass/moving average or Kalman filtering operation followed by a power spectral density analysis, followed by a normalization operation, a biometric signal statistics parameter amalgamation operation, and a biometric signal statistics parameter estimation operation creating biometric statistics estimate vectors.
